(19) United States
(12) Patent Application Publication
(10) Pub. No.: US 2023/0377157 A1
(43) Pub. Date: Nov. 23, 2023

(54) SEGMENTATION IN MULTI-ENERGY CT DATA

(71) Applicant: MARS BIOIMAGING LIMITED, Christchurch (NZ)

(72) Inventors: Philip Howard BUTLER, Christchurch (NZ); Anthony Philip Howard BUTLER, Christchurch (NZ); Praveenkumar KANITHI, Abu Dhabi (AE); Niels DE RUITER, Christchurch (NZ); Robert LINDEMAN, Christhurch (NZ)

(21) Appl. No.: 17/913,532

(22) PCT Filed: Mar. 22, 2021
(86) PCT No.: PCT/NZ2021/050046

§ 371 Date: Sep. 22, 2022

(30) Foreign Application Priority Data
Mar. 23, 2020 (NZ) 762875

(51) Int. Cl. G06T 7/11; G06V 10/764; G06V 10/762; G06V 10/776; G06T 11/00; G06V 20/70; G06T 7/194

(52) U.S. Cl. CPC G06T 7/11; G06V 10/764; G06V 10/763; G06V 10/776; G06T 11/001; G06V 20/70; G06T 7/194; G06T 2207/10081; G06T 2207/20092; G06V 10/464

(57) ABSTRACT

Segmentation of multi-energy CT data, including data in three or more energy bands. A user is enabled to input one or more region indicators in displayed CT data. At least some data is labelled based on the region indicators. Feature vectors are created for at least some data elements, which are then classified based on the labelled data elements and feature vectors. Feature vectors may be constructed using a Bag of Features or similar process. Classification may be performed using a Support Vector Machine classifier or other machine learning classifier.

### FIELD OF INVENTION

This invention relates to the segmentation of data, particularly but not exclusively data produced using a multi-energy computed tomography (CT) scanning system.

### BACKGROUND TO THE INVENTION

Multi-energy computed tomography (CT) is an x-ray imaging modality which produces 3D images of the inside of objects. CT scanners use polychromatic x-ray sources which emit a full rainbow or spectrum of x-rays with various ‘colours’ (x-ray energies). In regular CT there is no distinction made between the different energies of x-rays. However, x-rays are absorbed differently by different materials in the body, and absorption also depends on x-ray energy. Multi-energy CT measures the absorption of x-rays in different energy ranges. Using the differences in x-ray absorption in these energy ranges it is possible to discriminate between (identify) and quantify various materials in a scanned subject.

Interactive image segmentation is a process of partitioning an image into disjoint and meaningful regions with the help of user guidance. Image segmentation in general plays an important role in a wide range of medical imaging applications and analysis tasks.

For example, image segmentation may allow the separation of trabecular and cortical bone to evaluate bone health, quantifying nano particles in a particular organ, tumour segmentation, region specific material quantification, etc. However, accurate and automatic segmentation of image data produced by a multi-energy CT system remains a challenging problem. Some segmentation solutions for dual-energy CT image data are known. However, little to no labelled data that is required for designing automatic segmentation solutions is available for the segmentation of data produced using multi-energy CT.

Several pre-clinical studies have shown that multi-energy CT is able to differentiate various types of tissues in the human body, as well as different contrast agents commonly used in CT imaging [1], [2]. However, present interactive segmentation methods are not designed to take advantage of the additional information provided by multi-energy CT images.

In the interactive image segmentation literature, both random walks [3] and graph cuts [4] model the image as a graph and learn a probability model from foreground and background scribbles given by the user. Grabcut [5] improved graph cuts by changing the interaction type from scribbles to a bounding box around the object. Geodesic star convexity [6] employed star convexity constraints on graph cuts to try to connect foreground objects and background objects. Geodesic graph cuts [7] is similar except that it uses the geodesic distance between pixels instead of geodesic star convexity constraints.

A range of methods use superpixels to perform fewer computations. For example, maximal-similarity region merging [8], which is an iterative region merging approach. However, most superpixel driven methods assume the input image to be a single channel or an RGB image, and use only low-level features to segment the image into the foreground and background. Region-based methods [9], [8], [10] use preliminary features such as histograms and mean intensities to represent superpixels. Due to the multi-channel nature of multi-energy CT datasets (e.g. the Applicant's datasets currently range from 4 to 8 channels), following the same strategy leads to an increase in the histogram length by the number of bins per channel times the additional number of channels, which in turn may cause curse of dimensionality problems.

Recently proposed deep learning based methods are shown to be very effective [11], [12]. They use convolutional neural networks to learn the object boundaries and shapes. However, in addition to requiring large training datasets, they suffer from limiting the algorithm to a fixed set of foreground objects.

For the purposes of this specification, the term “multi-energy CT” refers to CT with 3 or more energy ranges or bins. This is distinct from dual-energy CT which uses only two energies.

### OBJECT OF THE INVENTION

It is an object of the invention to provide a computer implemented image segmentation method suitable for use with data produced using a multi-energy CT system.

Alternatively, it is an object of the invention to provide a computer implemented image segmentation method incorporating a bag of features method.

Alternatively, it is an object of the invention to at least provide the public with a useful choice.

### SUMMARY OF THE INVENTION

In one aspect the invention may provide a computer-implemented method for segmentation of multi-energy CT data, the multi-energy CT data including data in three or more energy bands, the method including: receiving in memory the multi-energy CT data; displaying the multi-energy CT data to a user; receiving user input of one or more region indicators for the displayed data; dividing the data into superpixels; labelling at least some of the superpixels based on the received region indicators; constructing feature vectors for at least some of the superpixels; based on the labelled superpixels and feature vectors, classifying the superpixels using a machine learning classifier; and segmenting the data into two or more regions based on the classification of the superpixels.

The machine learning classifier may be a Support Vector Machine.

The segmented data may be displayed to a user. The segmented data may be stored in memory.

Dividing the data may be achieved using Simple Linear Iterative Clustering. The superpixels may be non-overlapping superpixels.

Classifying the superpixels may include classifying global superpixel level descriptors into different classes.

Constructing feature vectors for at least some of the superpixels may be performed using a compact coding process.

Constructing feature vectors for at least some of the superpixels may be performed using a Bag of Features, Bag of Words, Fisher Vectors or Vector of Locally Aggregated Descriptors (VLAD) process.

Constructing feature vectors for at least some of the superpixels may include encoding and pooling the feature vectors. Encoding may include clustering labelled superpixels to create a codebook of visual words. Encoding may be performed using clustering techniques selected from k-means clustering, fuzzy clustering or gaussian mixture models. Pooling may include generating feature vectors using the codebook of visual words and augmented data from each superpixel.

A plurality of feature vectors may be constructed per superpixel.

Construction of feature vectors for a superpixel may include randomly sampling pixels from that superpixel.

One or more post processing steps may be performed, selected from smoothing object boundaries, and correcting misclassifying object regions.

If the segmentation of the image is determined to be unacceptable, the method may include receiving user input of one or more further region indicators for the displayed data; labelling at least some of the superpixels based on the received further region indicators; based on the labelled superpixels and feature vectors, reclassifying the superpixels using the linear Support-Vector Machine; and resegmenting the image into two or more regions based on the reclassification of the superpixels.

The image may be augmented with one or more of: texture information, horizontal gradient information and vertical gradient information.

The texture information may be generated from semi-local pixel information.

The feature vectors may be all of the same length.

In another aspect a computer-implemented method for segmentation of multi-energy CT data, the multi-energy CT data including data in three or more energy bands, may include: receiving the multi-energy CT data in memory; displaying the multi-energy CT data to a user; receiving user input of one or more region indicators for the displayed data; dividing at least some of the multi-energy CT data into superpixels; labelling at least some of the superpixels based on the received region indicators; constructing feature vectors for at least some of the superpixels; and based on the feature vectors and labelled superpixels, segmenting the image into two or more regions.

In a further aspect, a computer-implemented method for segmentation of multi-energy CT data, the multi-energy CT data including a plurality of data elements in three or more energy bands, may include receiving the multi-energy CT data in memory; displaying the multi-energy CT data to a user; dividing the multi-energy CT data into clusters of data elements; receiving user input of one or more region indicators for the displayed data; based on the received region indicators, labelling one or more of the clusters of data elements; constructing feature vectors for at least some clusters of the data elements; based on the feature vectors and labelled clusters of data elements, classifying the clusters of data elements; and segmenting the image into two or more regions based on the classification of the clusters of data elements.

The data elements may be pixels or voxels.

The clusters of data elements may be superpixels or supervoxels.

A multi-energy CT method may include performing a CT scan using a multi-energy CT system using three or more energy bands, to produce multi-energy CT data; and segmenting the multi-energy CT by any of the methods set out above.

In another aspect, a computer-implemented method for segmentation of image data, the image data including a plurality of data elements, may include receiving the image data in memory; displaying the image data to a user; receiving user input of one or more region indicators for the displayed image; based on the received region indicators, labelling one or more of: one or more of the data elements; and one or more clusters of the data elements; using a Bag of Features, Bag of Words, Fisher Vectors or Vector of Locally Aggregated Descriptors process. to construct feature vectors for one or more of: at least some of the data elements; and at least some clusters of the data elements; based on the feature vectors and labelled data elements and/or labelled clusters of data elements, segmenting the image into two or more regions.

A multi-energy CT data segmentation system, may include: memory arranged to store multi-energy CT data including data in three or more energy bands; a display arranged to display the multi-energy CT data to a user; a user input device arranged for user input of one or more region indicators for the displayed data; and a processor arranged to: divide at least some of the multi-energy CT data into superpixels; label at least some of the superpixels based on the received region indicators; construct feature vectors for at least some of the superpixels; and based on the labelled superpixels and feature vectors, classify the superpixels using a linear Support-Vector Machine; and segment the data into two or more regions based on the classification of the superpixels.

A multi-energy CT system, may include: a multi-energy CT scanner configured to scan a subject to produce multi-energy CT data including data in three or more energy bands; memory arranged to store the multi-energy CT data; a display arranged to display the multi-energy CT data to a user; a user input device arranged for user input of one or more region indicators for the displayed data; and a processor arranged to: divide at least some of the multi-energy CT data into superpixels; label at least some of the superpixels based on the received region indicators; construct feature vectors for at least some of the superpixels; and based on the feature vectors and labelled superpixels, segment the image into two or more regions.

In another aspect, a computer-implemented method for segmentation of an image produced using a multi-energy CT system using three or more energy bands, may include:
a) scanning an object using a multi-energy CT system using three or more energy bands;
b) storing the image data set of a) on a storage medium;
c) sending image data to be displayed on a graphical user interface;
d) receiving a plurality of strokes/markings generated by user input to the digital image to label image sections as a foreground or background;
e) dividing the image into non-overlapping superpixels and labelling as foreground or background based on strokes/markings of d);
f) constructing and assigning feature vectors for said superpixels using a compact coding process;
g) classifying the global superpixel level descriptors into different labels using a linear Support-Vector Machine;
h) segmenting the image into foreground and background based on the classification of g); and
i) presenting the segmented image to an interface.

Superpixel generation may be achieved using Simple Linear Iterative Clustering.

Labelled superpixels may be represented as

{(Sᵢ,Lᵢ)}ᵢ₌₁^{N},Lᵢ∈{0,1, . . . (M−1)} (1)

where Sᵢ denotes the i^{th} labelled superpixel, and Lᵢ denotes its corresponding label and M is the maximum number of classes Lᵢ takes its value from.

The compact coding process may include encoding and pooling the feature vectors.

One or more post processing steps may be performed, selected from smoothing object boundaries, correcting misclassifying object regions using connected component analysis.

Actions d)-i) may be repeated to obtain improved segmentation accuracy.

The compact coding process may be selected from a Bag of Features, Bag of Words, Fisher Vectors or Vector of Locally Aggregated Descriptors (VLAD).

The method may include creating an augmented image to improve classification accuracy. Creating an augmented image may include augmenting the image with texture and horizontal and vertical gradient information to improve segmentation.

The texture information may be generated from semi-local pixel information.

Encoding may include clustering labelled superpixels to create a codebook of visual words.

Encoding may be performed using clustering techniques selected from k-means clustering, fuzzy clustering or gaussian mixture models.

Pooling may include generating feature vectors using the codebook of visual words and augmented data from each superpixel.

Encoding and pooling may be completed using a Vector of Locally Aggregated Descriptors (VLAD) framework.

The number of feature descriptors/vectors generated per superpixel may be increased to artificially produce more training data for classification at h).

A system for segmentation of an image produced using a multi-energy CT system using three or more energy bands, may include:
a) a means for scanning an object using a multi-energy CT system to produce a set of image data;
b) a means for storing the set of image data;
c) a means for sending the set of image data to a graphical user interface;
d) a means for visually displaying the image data;
e) a means for receiving a plurality of strokes/markings generated by user input;
f) a means for dividing the image into non-overlapping superpixels and labelling as foreground or background based on strokes/markings of d);
g) a means for constructing and assigning feature vectors for said superpixels using a compact coding process;
h) a means for classifying the global superpixel level descriptors into different labels using a linear Support-Vector Machine;
i) a means for segmenting the image into foreground and background based on the classification of h); and
j) a means for presenting the segmented image to an interface.

A system for segmentation of an image produced using a multi-energy CT system using three or more energy bands, may include:
a) a multi-energy CT scanning system for scanning an object to produce a set of image data;
b) a data storage medium for storing the set of image data;
c) a transmitter for sending the set of image data to a graphical user interface;
d) a graphical user interface for visually displaying the image data;
e) a processor for receiving a plurality of strokes/markings generated by user input;
f) a processor for dividing the image into non-overlapping superpixels and labelling as foreground or background based on strokes/markings of d);
g) a processor for constructing and assigning feature vectors for said superpixels using a compact coding process;
h) a linear Support-Vector machine for classifying the global superpixel level descriptors into different labels;
i) a processor for segmenting the image into foreground and background based on the classification of h); and
j) an interface for presenting the segmented image to a user.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1 shows the method of interactive image segmentation of the input image using a bag of features approach in a preferred embodiment of the invention;

FIG. 2 shows a computer implemented method of interactive image segmentation of FIG. 1 in one embodiment of the invention;

FIG. 3 shows an illustration of the construction of the augmented image for an RGB image in one aspect of the invention;

FIG. 4 shows two stages in constructing a feature vector using the bag of features approach in one aspect of the invention;

FIG. 5 shows an example visual comparison of the segmentation performance on a lamb dataset;

FIG. 6 shows an example visual comparison of segmentation performance on a knee dataset with varying noise levels;

FIG. 7 shows a representation of one embodiment of a system for implementing any one or more of the methods shown in FIG. 1 or FIG. 2;

FIG. 8 shows a representation of an alternative embodiment of a system for implementing any one or more of the methods shown in FIG. 1 or FIG. 2.

### BRIEF DESCRIPTION OF PREFERRED EMBODIMENTS OF THE INVENTION

The invention disclosed herein relates to the interactive segmentation of multi-energy CT data sets. Segmentation may be performed using a bag of features method or other compact coding process. The invention also relates to one or more CT scanning systems, software facilities, computer program products, computer systems or computer implemented methods for the interactive segmentation of multi-energy CT data sets.

The Applicant's segmentation method may be used, for example, for segmenting or isolating a particular region in multi-energy CT data. In CT scans on human or animal subjects, the region to be isolated may be a particular organ, part of an organ, bone, part of a bone, tumour or other anatomical region.

In literature, most existing segmentation methods are limited to performing a specific task or tied to a particular imaging modality. Therefore, when applying generalized methods to datasets produced using multi-energy CT, the additional energy information acquired from the CT scanner cannot be sufficiently utilized.

Described herein is a new approach that circumvents this problem by effectively aggregating the data from multiple channels. The method solves a classification problem to get the solution for segmentation.

Starting with a set of labelled pixels, the data may be partitioned using superpixels.

Then, a set of local descriptors, extracted from each superpixel, may be encoded into a code-book and pooled together to create a global superpixel level feature vector (bag of features representation).

The vector of locally aggregated descriptors may be employed as the encoding/pooling strategy, as it is efficient to compute and leads to good results with simple linear classifiers.

A linear-Support Vector Machine may be used to classify the superpixels into different labels.

The proposed method was evaluated on multiple datasets produced using a multi-energy CT scanner. Experimental results show that the method disclosed herein achieved an average of more than 10% increase in the accuracy over other known methods.

In one embodiment, the method may specifically focus on creating high-level, fixed-length feature vectors for superpixels using a bag of features approach. Generating feature vectors in this way not only aggregates information from all spectral channels, but also gives us the ability to control the feature vector lengths.

Data augmentation strategies are also proposed to increase the number of feature vectors to have better classification accuracy.

Upon evaluating against a set of state-of-the-art methods, the method described herein achieves higher segmentation accuracy compared to other traditional methods.

In the example below, we solve a classification problem to interactively segment spectral CT images into two regions (‘foreground’ and ‘background’) using a bag of features approach.

### Materials and Methods

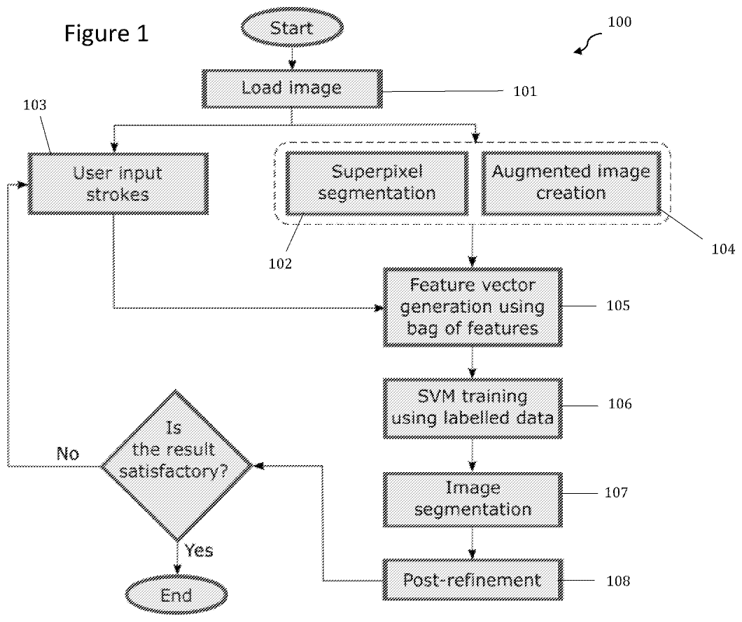

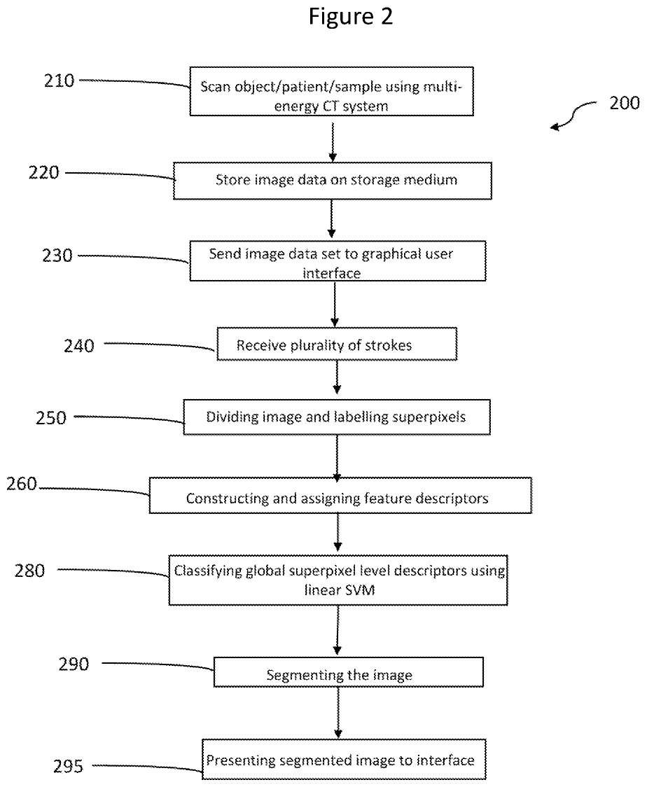

The method 100 of the proposed segmentation process for a single 2D slice of multi-energy CT data is depicted in FIG. 1 and is summarised below: FIG. 2 outlines the method as a series of computer implemented steps.

In method 100 (FIG. 1), an image may be processed into a set of superpixels while the user draws strokes to label the foreground and background. The superpixels containing the user's strokes are then labelled as belonging to the stroke's class (from now on called labelled superpixels). For the purposes of this specification, the terms ‘label’, ‘labelling’ etc require that the relationship between data element (e.g. superpixels) containing strokes and the class of those strokes is explicitly known. That relationship may be stored/registered in any suitable manner.

These labelled superpixels may then be studied to generate feature vectors using a bag of features approach. For better accuracy while computing feature vectors, as an optional step an augmented image may be generated using high level features by adding texture and gradient maps. Then, image segmentation may be performed using a linear Support Vector Machine (SVM) or other suitable machine learning based classifier, with the help of the labelled superpixels. As shown in FIG. 1, if the user is not satisfied with the final result, the result can be further refined by adding more strokes to the foreground and/or the background.

### A. Superpixel Generation

Scan data from a multi-energy CT scanner may be stored in memory. An image that is derived from the CT scan data may be loaded 101 and divided into superpixels 102. The image 101 may generally be, or be based on, a 2D slice of multi-energy CT data.

Superpixels are a group of pixels merged together into meaningful sub image regions. Unlike pixels in a rigid pixel grid, a superpixel can be of any size and shape. They capture the redundancy of pixels in sub image regions, and provide a convenient form to extract features. Overall, having fewer data points to process reduces the computational requirements for subsequent processing tasks.

One major advantage of superpixels is their robustness towards noise in the image. Just like any other medical datasets, multi-energy CT datasets often contain noise and at times image artifacts [14]. The fact that superpixels are used for segmentation rather than a single pixel may smooth out some of the inherent noise present in the image.

There are many approaches to generate superpixels. For example, Simple Linear Iterative Clustering (SLIC) [15] may be used. Other suitable methods of generating superpixels may include: derivatives of SLIC (e.g. manifold SLIC (MSLIC)); Gaussian generative models and derivatives thereof; Normalized cuts; Turbopixels; EWVCT; Vcells; BGD (bilateral geodesic distance); LRW (lazy random walk); DBSCAN and other available superpixel generation methods.

These methods may provide varying performance and quality of superpixel generation and therefore of the resulting segmentation.

SLIC is an adaptation of the k-means [16] clustering approach to efficiently generate superpixels. Previous research has shown that SLIC has a better tradeoff between computational complexity, accuracy, and boundary compactness than most state-of-the-art algorithms [17] and [18].

For the implementation of interactive segmentation, the image is displayed to a user. The user inputs one or more region indicators, e.g. a set of strokes 103 via any suitable user input, including e.g. touch screens, pointer devices, computer mouse etc, to indicate the pixels that belong to a particular region or class. Strokes, scribbles or markings may be entered onto an image on a graphical interface by an informed user to indicate foreground and background regions in an image.

As the image is already divided into superpixels, each superpixel may be considered labelled if any one pixel inside it is marked by a stroke. This assumption may be considered acceptable as superpixels tend to contain pixels that are homogeneous in nature. When a superpixel is labelled, all of the pixels contained within it may be automatically labelled as well. Let the number of labelled superpixels be N, then all the labelled superpixels can be represented as

{(Sᵢ,Lᵢ)}ᵢ₌₁^{N},Lᵢ∈{0,1, . . . (M−1)} (1)

where Sᵢ denotes the i^{th} labelled superpixel, and Lᵢ denotes its corresponding label. M is the maximum number of classes Lᵢ takes its value from (M=2 in this example).

### B. Creating an Augmented Image

The multi-energy CT image may be augmented 104 with texture and horizontal and vertical gradient information to improve segmentation.

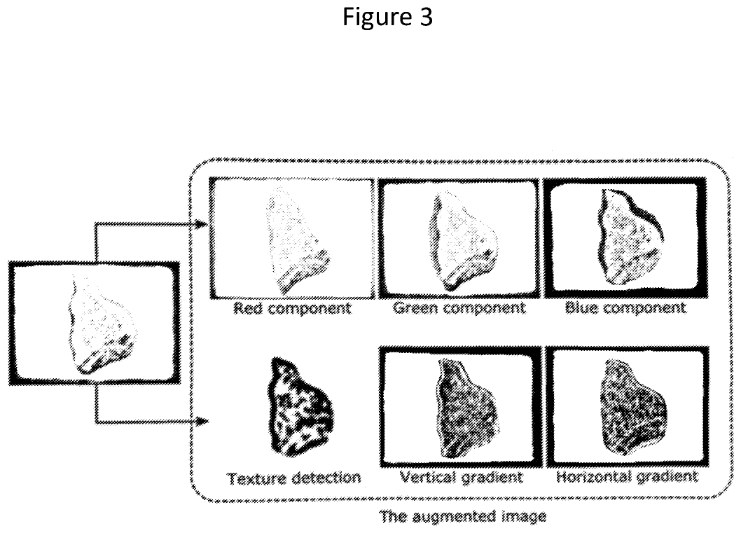

FIG. 3 provides an illustration of one procedure of constructing an augmented image 104 (FIG. 1) from the input. In the example of FIG. 3, an RGB image is taken as an input where the red, green and blue channels are analogous to multiple energy channels in multi-energy CT images. Computing gradient information is straight forward, with the average grayscale image of all the energy channels used for this purpose. However, there are several ways to approach texture synthesis [20], [21], [22]. The framework developed in [20] may be used for our purpose as it considers data from multiple channels to compute a single texture descriptor. We describe the basics of this framework below.

Texture features based on semi-local image information: It is impossible to quantify texture using information from a single pixel, since it is semi-local by nature. Semi-local information consists of a close neighbourhood around the current pixel whereas local information includes just the position and value of the pixel in consideration. This can also be called a patch.

Consider for instance a 2D grayscale image I={Iᵢⱼ:(i,j)∈Ω}. It can be viewed as a function I(x,y):Ω→R.

To denote the semi-local information, a square patch P_{xy}(I) of size n×n (an odd positive integer) centered at pixel (x, y) may be extracted.

$${𝒫}_{xy} ( I ) = \{ I ( x + k , y + l ) : k , l = - \frac{( n - 1 )}{2} , … , \frac{( n + 1 )}{2} \} \qquad ( 2 )$$

It may be considered that the given input image is obtained by discretizing a differentiable surface that allows the use of some efficient tools from classic differential geometry.

Sochen et al. [24] proposed to represent the image as Remannian manifolds embedded in a higher dimensional space. For example, a 2D gray image I:R²→R, can be viewed as a surface (denoted by Σ) with local coordinates (x, y) embedded in R³ by a mapping M_{xy}(I)→(x,y,I(x,y)).

Using formula (2) above, this manifold based representation can be extended to support semi-local information at location (x, y)

[symbol]

(I)→(x, [symbol] , [symbol] (I)) (3)

In the above mapping, the first two components indicate local information and the semi-local information is included in the form of a patch P_{xy}(I). From the theory of differential geometry, the area of an element on the surface M_{xy}(I) is defined as

$$‖ \frac{\partial ℳ}{\partial x} \times \frac{\partial ℳ}{\partial y} ‖$$

$$Where \frac{\partial ℳ}{\partial x} = ( 1 , 0 , \frac{\partial 𝒫}{\partial x} ) and \frac{\partial ℳ}{\partial y} = ( 1 , 0 , \frac{\partial 𝒫}{\partial y} )$$

The partial derivatives ∂P/∂x and ∂P/∂y can be computed using forward differences since the image is discrete. Using this area definition, the rate of change of the area of a surface element is defined as

$$G = ‖ \frac{\partial ℳ}{\partial x} \times \frac{\partial ℳ}{\partial y} ‖ = \sqrt{( 1 + {( \frac{\partial 𝒫}{\partial x} )}^{2} ) ( 1 + {( \frac{\partial 𝒫}{\partial y} )}^{2} ) - {( \frac{\partial 𝒫}{\partial x} \cdot \frac{\partial 𝒫}{\partial y} )}^{2}} \qquad ( 4 )$$

In the regions where texture is present, the intensity variations in the local neighborhood cause the corresponding surface to change with different G values. For images with more than one channel, (4) can be extended to support multiple channels. Let I=(I₁, I₂, . . . , I_{d}), where d represents the number of channels. From formula (3), the corresponding manifold based representation can be modified as M_{xy}(I)→(x, y, P_{xy}(I₁), . . . , P_{xy}(I_{d})). The final rate of area change for a multi-channel image becomes:

$$G = \sqrt{( 1 + {\sum }_{i = 1}^{d} {( \frac{\partial 𝒫 ( {I}_{i} )}{\partial x} )}^{2} ) ( 1 + {\sum }_{i = 1}^{d} {( \frac{\partial 𝒫 ( {I}_{i} )}{\partial y} )}^{2} )} - {\sum }_{i = 1}^{d} {( \frac{\partial 𝒫}{\partial x} \cdot \frac{\partial 𝒫}{\partial y} )}^{2} \qquad ( 5 )$$

The texture descriptor T is finally defined as T=exp(−G²). After applying the texture descriptor T to the input image I, the resulting single channel image can be denoted by Iₜ={tᵢⱼ:(i,j)∈Ω}. In a given textured region, pixel T values within that region will be similar. Across the image there may be different texture regions, where the value of T may change. In addition to the Gaussian smoothing, the semi-local nature of this texture descriptor over multiple channels makes it more robust to the noise compared to other texture descriptors [22].

While one example of use of augmentation has been described, the skilled reader will understand that other methods of image augmentation may be suitable. In particular, methods that augment the CT data in order to improve segmentation may be used. Augmentation with texture information may be used. Augmentation with horizontal and/or vertical gradient information may be used.

### C. Feature Vector Extraction

Returning to FIG. 1, following superpixel segmentation and augmented image creation, feature vectors may be generated using a bag of features approach 105. Other compact coding processes may also be suitable, including Bag of Words (generally considered a synonym for Bag of Features in image analysis), Fisher Vectors or Vector of Locally Aggregated Descriptors (VLAD). VLAD may be considered an extension of a Bag of Features approach.

Feature vectors give a meaningful and quantitative representation to superpixels. Having discriminative feature vectors for the foreground and background can lead to accurate segmentation. As mentioned before, colour histograms or mean intensities might not be able to distinguish between foreground and background objects with similar visual properties or cause the curse of dimensionality issue. To avoid such situations, the Applicant's method may use a bag of features based approach to construct feature vectors of uniform or fixed length [25].

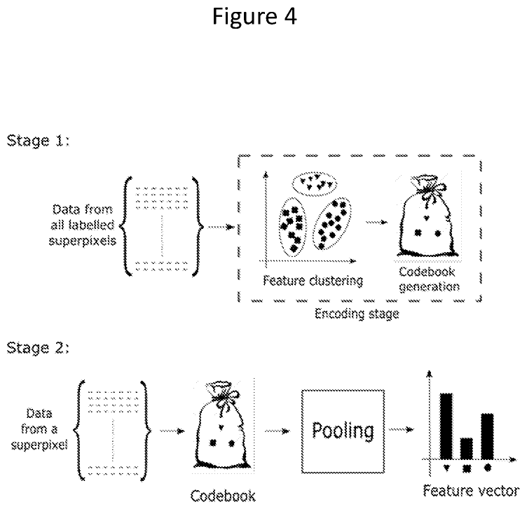

FIG. 4 outlines the bag of features process in two stages: encoding and pooling. In the encoding stage (Stage 1, FIG. 4), all the labelled information may be clustered together to generate a codebook C=(c₁, c₂, . . , c_{K}) of K visual words. Each row of the data used in this stage represents the augmented information at a pixel within all the labelled superpixels. Conventionally, this is also called a local descriptor (p).

In the pooling stage (Stage 2, FIG. 4), the same codebook C and the augmented data from each superpixel are used to generate feature vectors.

J\acute{ }egou et al. [26] have shown that bag of features can have multiple variations based on how the encoding and pooling processes are performed. For example, encoding can be done using different clustering techniques such as k-means clustering, fuzzy clustering or gaussian mixture models, etc. The simplest form of pooling is constructing a histogram using the information from the codebook.

In one proposed method, Vector of Locally Aggregated Descriptors (VLAD) framework is used to do encoding and pooling [27], [26].

In VLAD, encoding involves using k-means clustering to generate the codebook C. This codebook is the basis for constructing global descriptors for all the superpixels (labelled and unlabelled included). During pooling, let each superpixel feature vector be represented by V={V_{(iK+j)}:i=0 . . . K−1, j=0 . . . d−1}, where i and j indexes the codebook and the local descriptor component respectively. In this case, local descriptor components are equal to the number of channels in the augmented image. Hence, each component of V is represented as

$${𝒱}_{( iK + j )} = \begin{matrix}\sum \\ p suchthat N N ( p ) = {c}_{i}\end{matrix} {p}_{j} - {c}_{i , j} \qquad ( 6 )$$

where pⱼ and c_{i,j} respectively denote the j^{th} component of the local descriptor p and its corresponding nearest visual word denoted by the nearest neighbour (NN).

The resulting feature vector V is L2-normalized (V:=V/∥V∥₂), and then subsequently power normalized (Vᵢ:=sign(νᵢ)\sqrt{|νᵢ|}) to enhance superpixel specific components, and to unsparsify the descriptors respectively [28]. While calculating the feature vectors, there is no hard and fast rule on how to select the number of clusters (K). However, using large values of K makes the descriptors unnecessarily large as well as sparse.

### D. Segmentation

As stated earlier, in our proposed approach the segmentation problem may be seen as a classification problem. Feature vectors corresponding to the labelled superpixels may be used to train a SVM classifier 106 [29]. The SVM may be a linear SVM. In general, any suitable machine learning based classifier, including any suitable supervised learning model and in particular any SVM may be used, including a multiclass SVM classifier, One-vs-One SVM or One-vs-Rest SVM.

In a standard approach, the classifier gets as many feature vectors as the number of labelled superpixels for training. However, in the current method, the number of feature vectors generated per superpixel may be increased to artificially produce more training data. Generation of feature vectors may be accomplished by randomly sampling pixels from each superpixel to generate a feature vector. This procedure may be performed several times on each superpixel to get multiple feature vectors per superpixel. All the feature vectors generated from a single superpixel share the same label. The random sampling may be done with replacement, so the total data from which the sampling is made is constant every time. This way, more data can be augmented for better classification accuracy. This process of increasing the number of training samples is also called data augmentation [30]. One of the reasons multiple feature vectors are generated from a single superpixel through random sampling is because of the fact that superpixels are nearly homogeneous over all the pixels within.

Once the training is over, the same codebook C is used to generate feature vectors for all the unlabelled superpixels. The trained SVM classifier uses these feature vectors to propagate labels to the entire image. Typically, this is how classification may be done to assign labels to the unlabelled samples. However, to give each superpixel its local context, the feature vectors may be modified as a weighted average of neighbourhood superpixels.

Feature vector ‘V(i)’ for a superpixel ‘i’ (Sᵢ) is defined as

$$𝒱 ( i ) = \frac{1}{{w}_{i}} {\sum }_{j ∈ \{ i , {𝒩}_{i} \}} {w}_{ij}^{r} {w}_{ij}^{d} 𝒱 ( j ) \qquad ( 7 )$$

$$where$$

$${w}_{ij}^{r} = exp ( - \frac{{‖ M ( i ) - M ( j ) ‖}_{2}^{2}}{{σ}_{r}^{2}} )$$

$${w}_{ij}^{d} = exp ( - \frac{dist ( i , j )}{{σ}_{d}^{2}} )$$

where, wᵢ completes the weighted average. Nᵢ is the set of neighbouring superpixels of Sᵢ, w^{r}ᵢⱼ is a range based weight based on the difference in mean intensities of the superpixels, and w^{d}ᵢⱼ is a domain based weight based on spatial distance.

σd, σr are smoothing parameters, M(⋅) denotes the mean intensity of the superpixel, and dist(i, j) is the Euclidean distance between the centroids (spatial) of superpixels i and j.

Once classification is performed, a few post-processing steps are needed to fine tune the result.

Once superpixels are classified into foreground and background using SVM, all the pixels within a superpixel are assigned the same class as the superpixel they belong to. This process is repeated for all the superpixels within the image to label/segment the entire image 107.

During one image segmentation process, all the pixels in the output image belonging to the same class are assigned the same intensity value (or some other distinction between the pixel classes may be made). For a two-class problem, this means that the resulting image is a binary image. However, this result may need some post-refinement 108 due to superpixel misclassification or imperfections during superpixel generation. The superpixel misclassification results in foreground objects to have background patches and vice versa due to similar visual properties the foreground and background objects share. To reduce this problem, connected component analysis may be used to discard such regions. It only preserves the regions with strokes, and other misclassified regions may be discarded, and merged into the background/foreground.

After the post-refinement, the end result may be displayed to the user for the feedback. If the user is satisfied with the generated segmentation result, segmentation is complete. If not, the user may give additional input strokes to refine the result. Once the additional strokes are given, the entire process may be repeated to segment the image using the old and new strokes.

During superpixel classification, there may be some false positives with similar visual properties. Connected component analysis is used to discard such region. Connected component analysis only preserves the regions with strokes, and other misclassified regions are discarded and merged into the background.

### Results

The example method described above generates feature vectors using bag of features and then segments the image with the help of these feature vectors and a classifier. This approach may work with any number of channels in a multi-energy CT system.

Results for two datasets are reported below, each with different amounts of noise. The performance of the method described is compared against state-of-the-art methods. These methods include Random Walk (RW) segmentation [3], Graph Cuts (GC) [4], Geodesic Star Convexity (GSC) prior for segmentation [6], Geodesic Graph Cuts (GGC) [7], Grabcut [5], and Maximal Similarity based Region Merging (MSRM) [8].

The following parameters were used in the current approach: the square patch size for calculating texture (n)=7×7, the number of visual words for VLAD (K)=16, and the range and domain based smoothing parameters (σᵣ and σ_{d}) were set to 1.8 and 1.3 respectively.

The same user input (as a set of input strokes) was provided to all methods in order to avoid any variations caused by input discrepancy. While additional strokes could improve the performance of either of the methods, they would also require additional user interaction, hence the analysis was carried out with a single fixed set of input strokes from the user. In all figures, input images have white and red coloured strokes to represent foreground and background objects respectively. The segmentation results from each of the tested method used in this paper were shown using blue contours. Similarly, green colour was chosen to denote the manually annotated ground truth. The ground truths for these datasets were manually annotated by members of a clinical team.

### A. Performance Analysis on the Lamb Dataset

For performance analysis, the first dataset used in our work is of a lamb chop (from now on referred to as the “lamb dataset”). This is an early multi-energy CT dataset and a challenging one to work with, due to the noise and the low contrast between lipid and water. The volume dimensions are 436×436×126 voxels, the cubic voxels are 0.093 mm wide, and four frames to represent four energy channels. In the lamb dataset, we chose three classes: lipid- like, water-like, and background. In the following text, the experiments demonstrate two cases in which 1) lipid-like objects are labelled as foreground and the rest as background, 2) water-like objects are labelled as foreground and the rest as background. Since the algorithm is mainly designed to work for 2D slices, the testing was done on multiple individual slices separately.

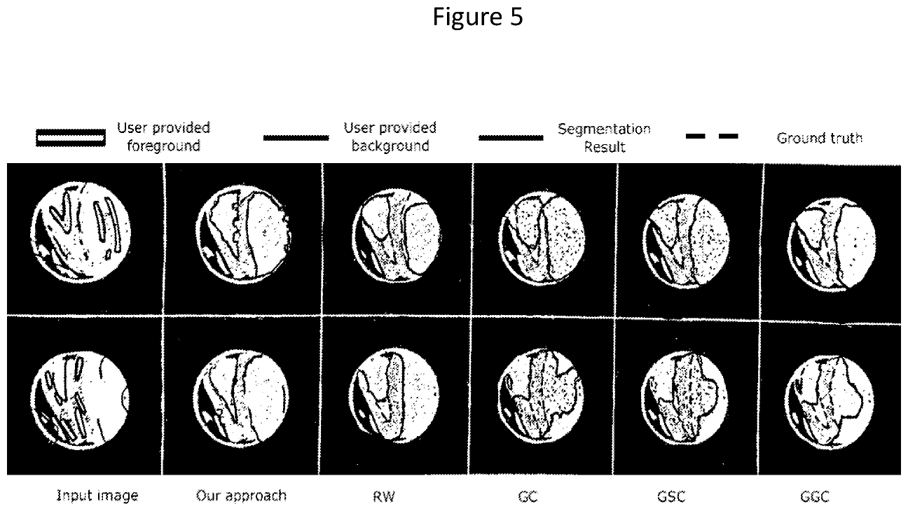

In FIG. 5, the top row shows the interactive segmentation of water-like objects from the lamb dataset. From the image, it shows that the proposed method is able to delineate the boundary comparatively better than the other methods. But none of the methods could cleanly segment the water-like material from the perspex tube in which the sample is kept.

The bottom row of FIG. 5 shows the same slice except this time the lipid-like material is labelled as the foreground. Qualitatively seen, the Applicant's segmentation is again superior to other methods, with the RW result being the closest in quality to the Applicant's approach. All other methods deviated on the right side into the background.

It can be observed that the boundaries have a slight block-like appearance at some places. The reason for their block-like appearance is due to the fact that SLIC starts off with square patches as superpixels and adjust their shape according to the nearby image content. Since the image in question doesn't have many features, most of the superpixels stayed square-like. After some of the superpixels were misclassified, these block boundaries become visible.

### B. Performance Analysis on the Knee Dataset

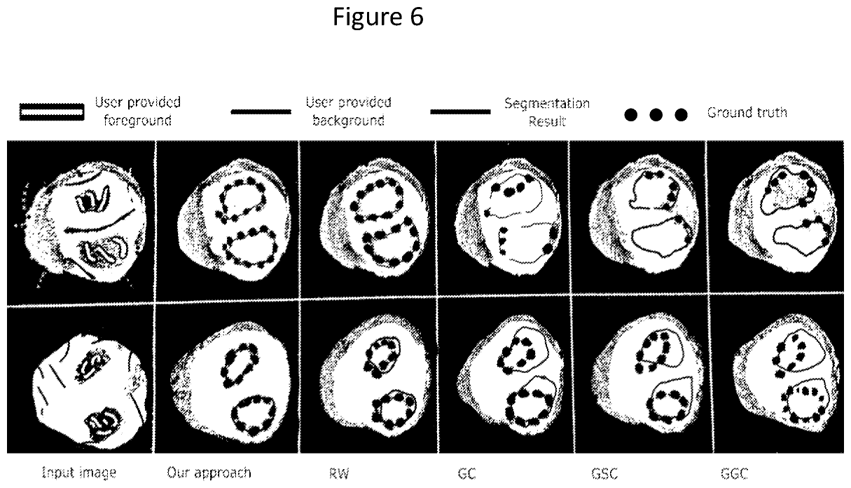

The second dataset used for our performance evaluation is of a knee doped with Hexabrix (from now on referred to as the “knee dataset”). Its volume dimensions are 1280×1280×192 voxels, the cubic voxels are 0.07 mm wide, and five energy channels. In this dataset, the object of interest is bone. Segmenting a bone might seem a trivial task, but it has its own challenges as will be demonstrated in FIG. 6. Similar to the Lamb dataset, the testing is done for a series of slices. Some of the chosen slices had ring artefacts to demonstrate the robustness of the system.

For this dataset, two cases are demonstrated to compare how the methods perform at different noise levels. The top row of FIG. 6 shows the results for segmenting bone in a slice with good contrast. It can be observed that the proposed algorithm has similar performance compared to the other methods. However, in the cases where the noise levels are high and contrast is limited, like in the bottom row of FIG. 6, the performance differs drastically from one algorithm to the other.

Even in these cases, the methods described herein produce images closer to the ground truth when compared to other methods.

### C. Quantitative Analysis of Results

The average performance of the proposed approach on the two datasets was quantified using accuracy, F1-score, and Intersection over Union (IoU) as statistical accuracy metrics. The manually delineated and segmented regions are denoted by I and G respectively.

Accuracy is defined as a ratio of the number of correctly classified pixels to all the pixels within an image. It is the most commonly used metric for performance evaluation, which indicates the degree of similarity between the segmentation result and their respective manually annotated ground truth.

Another similarity index used for this evaluation is called F1-score (the Dice's coefficient). Given I and G, it is defined as;

$$F 1 - score = \frac{2 \vert I ⋂ G \vert}{\vert I \vert + \vert G \vert} \qquad ( 8 )$$

where |I| and |G| are the cardinalities of the sets I and G respectively (i.e. the number of elements in each set). F1-score or the Dice's coefficient not only measures the similarity between I and G, but also considers the foreground and background class balance. It computes the ratio of the overlapping region between the ground truth foreground and the foreground segmented by the algorithm to the combined foreground size.

The overlapping area of segmented and ground truth is quantified using IoU or the Jaccard measure. It is defined as the ratio of the number of pixels within the area of intersection to the number of pixels within the union of regions.

$$IoU = \frac{\vert I ⋂ G \vert}{\vert I ⋃ G \vert} \qquad ( 9 )$$

TABLE I
A PERFORMANCE COMPARISON OF THE PROPOSED
APPROACH AGAINST OTHER TRADITIONAL
INTERACTIVE SEGMENTATION ALGORITHMS.

| Method | Accuracy(%) | F1 score(%) | IoU(%) |
|---|---|---|---|
| GC | 95.10 ± 2.69 | 78.53 ± 12.73 | 66.29 ± 16.63 |
| RW | 96.37 ± 1.91 | 83.19 ± 7.37 | 71.86 ± 10.67 |
| GSC | 94.13 ± 1.48 | 73.98 ± 10.66 | 59.73 ± 13.35 |
| Grabcut | 88.07 ± 1.88 | 62.90 ± 6.11 | 46.14 ± 6.56 |
| MSRM | 90.52 ± 2.32 | 63.38 ± 10.84 | 51.41 ± 12.74 |
| GGC | 94.31 ± 1.47 | 74.49 ± 10.54 | 60.35 ± 13.26 |
| Proposed | 97.91 ± 0.82 | 89.07 ± 5.08 | 80.63 ± 7.92 |

Table I outlines the average performance of the algorithms for a given input over the two datasets. All the algorithms were given the same input except grabcut, which takes a bounding box as an input.

The bold representation of the score indicates the best performance in each metric. Performance wise, RW comes close to the proposed approach, but still has a significant difference. Another thing to notice here is that the standard deviation, which reveals the consistency of the results over large number of images. The proposed method has a relatively small standard deviation compared to the other methods, which indicates its stability over different images.

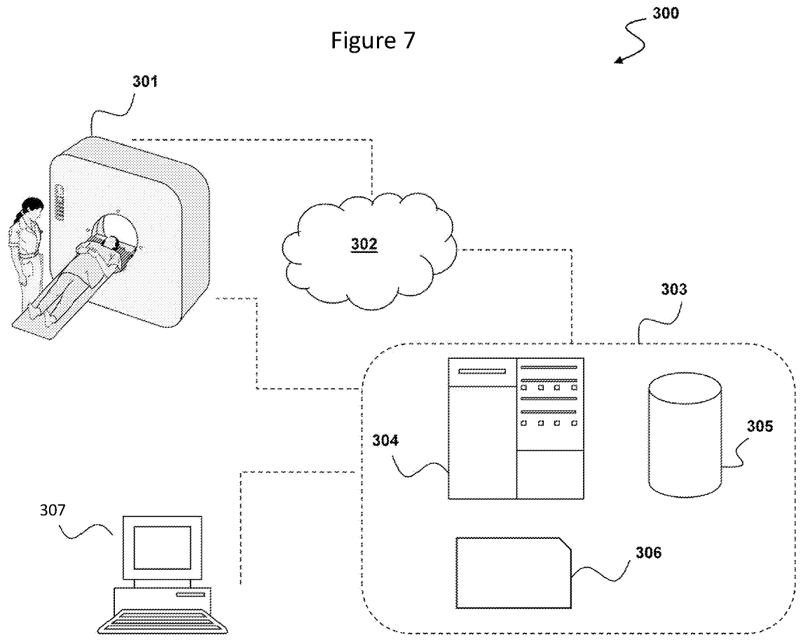

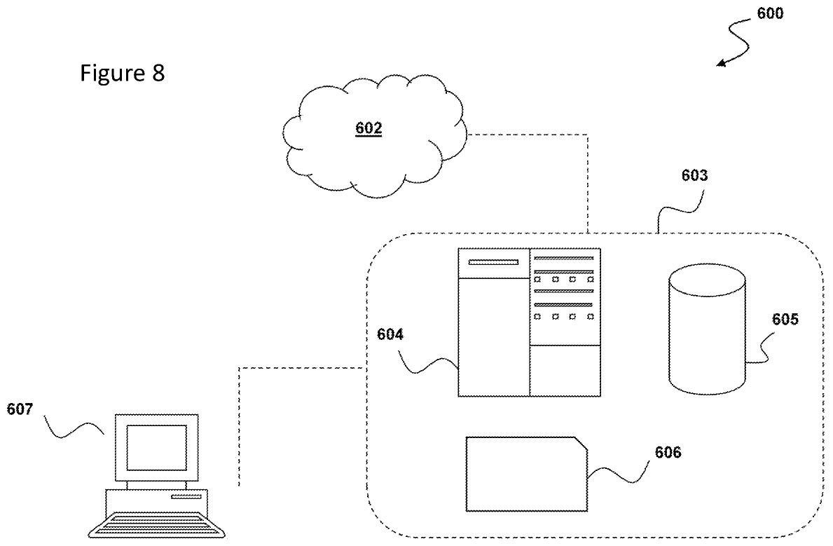

The computer implemented methods of the present invention are outlined as method 200 in FIG. 2 in one preferred embodiment and may be operated using the systems as exemplified in FIGS. 7 and 8.

In use with method 200 of FIG. 2, the system 300 of FIG. 7 includes a multi-energy CT imaging scanner 301. Multi-energy CT images are taken of a subject (e.g. an object/sample or patient of interest) 210 and the image data set delivered via communications network 302 to a user interface 307 via computer system 303. Computer system 303 includes processor 304, data storage medium 305 and server 306.

Processor 304 receives the image data set from communications network 302 and implements the processing steps of the methods of the present invention together with data storage medium 305 and user input received from interface 307. Server 306 serves and receives data between computer system 303 to user interface 307.

FIG. 8 shows an alternate system 600 of the present invention, where multi-energy CT image data is received over a communications network 602 by a processor 604 within computer system 603. Multi-energy CT data is received over communications network 602 from an external source via known communications protocols for example, email, internet, FTP or HTML.

As with system 300 computer system 603 includes processor 604, data storage medium 605 and server 606. Processor 604 receives the image data set from communications network 602 and implements the processing steps of the methods of the present invention together with data storage medium 605 and user interface 607. Server 606 serves data to and from computer system 603 to interface 607.

The various operations of methods described above may be performed by any suitable means capable of performing the operations, such as various hardware and/or software component(s), circuits, and/or module(s). Generally, any operations illustrated in the Figures may be performed by corresponding functional means capable of performing the same or equivalent operations.

The various illustrative modules and algorithm steps described in connection with the embodiments disclosed herein may be implemented as electronic hardware, computer software, or combinations of both. To clearly illustrate this interchangeability of hardware and software, various illustrative components, modules, and steps have been described above generally in terms of their functionality. Whether such functionality is implemented as hardware or software depends upon the particular application and design constraints imposed on the overall system. The described functionality may be implemented in varying ways for each particular application, but such implementation decisions should not be interpreted as causing a departure from the scope of the embodiments of the invention.

The steps of a method or algorithm and functions described in connection with the embodiments disclosed herein may be embodied directly in hardware, in a software module executed by a processor, or in a combination of the two. If implemented in software, the functions may be stored on or transmitted over as one or more instructions or code on a tangible, non transitory computer-readable medium.

A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD ROM, or any other form of storage medium known in the art.

A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blu ray disc where disks usually reproduce data magnetically, while discs reproduce data optically with lasers.

Combinations of the above should also be included within the scope of computer readable media. The processor and the storage medium may reside in an ASIC. The ASIC may reside in a user terminal. In the alternative, the processor and the storage medium may reside as discrete components in a user terminal.

The examples above relate primarily to segmentation into two regions (which may be considered ‘foreground’ and ‘background’ or may be given some other label). However, the invention may also extend to segmentation into more than two regions. For example, this may be achieved by a user identifying three or more regions, labelling the superpixels accordingly, and segmenting by similar methods to those described above. Alternatively, the process may be repeated with different foreground targets each time to ultimately yield three or more regions.

Further, while the invention has been described primarily in relation to a two dimensional ‘slice’ of multi-energy CT data, it may be possible to extend the method to segmentation of three dimensional data. For example, an initial segmentation on 2D data may be extended into 3D data. Alternatively, multiple segmentations may be performed in different 2D ‘slices’ before extension into 3D data. Alternatively, the method may be performed on voxels and supervoxels rather than pixels and superpixels. In general, pixels and voxels may be considered data elements, while superpixels and supervoxels may be considered clusters of data elements (notwithstanding that the smallest possible superpixel is a single pixel, and the smallest possible supervoxel is a single voxel).

Extending the above example to supervoxels may introduce complexity that significantly slows the process. Repeating the above process slice by slice may therefore be a preferred approach to segmentation in three dimensions.

Further, if a 2D segmentation is to be extended to 3D, the initial 2D segmentation may be an interactive segmentation based on user-input markings, strokes or similar. Segmentations of further slices or otherwise extending the 2D segmentation into 3D may be performed without further user-input of markings/strokes etc. This may be done using the labels and vectors already determined, or further strokes, markings etc may be automatically created.

The segmented data resulting from the Applicant's process may be displayed to a user on any suitable display.

The segmented data may also be applied to the original data to extract all pixels belonging to a class into its own image. For example, if the original image is of a patient's chest, the segmentation result may be applied to create a new image with just one structure of interest (e.g. the heart) and a second new image with everything except that structure of interest (e.g. the heart).

Alternatively, the segmentation data may be fed directly into visualisation/analysis algorithms to include or exclude parts of the data.

Annotations and/or metadata for the original image may be created based on the regions specified by the segmentation results.

The segmentation results, and/or any derivative images/data/analysis results may be saved to any suitable storage.

While described primarily in relation to multi-energy CT scanners, the invention may also be applicable in other scanners, including scanners that provide multi-energy CT data, such as hybrid scanners (e.g. MRI and multi-energy CT) or phase-contrast CT scanners providing multi-energy CT data,

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

### REFERENCES

[1] M. Moghiseh, R. Aamir, R. K. Panta, N. de Ruiter, A. Chernoglazov, J. Healy, A. Butler, and N. Anderson, “Discrimination of multiple high-z materials by multi-energy spectral ct-a phantom study,” JSM Biomed Imaging Data Pap, vol. 61, p. 1007, 2016.
[2] K. Rajendran, C. L\ddot{ }obker, B. S. Schon, C. J. Bateman, R. A. Younis, N. J. de Ruiter, A. I. Chernoglazov, M. Ramyar, G. J. Hooper, A. P. Butler, et al., “Quantitative imaging of excised osteoarthritic cartilage using spectral ct,” European radiology, vol. 27, no. 1, pp. 384-392, 2017.
[3] L. Grady, “Random walks for image segmentation,” IEEE transactions on pattern analysis and machine intelligence, vol. 28, no. 11, pp. 1768-1783, 2006.
[4] Y. Y. Boykov and M.-P. Jolly, “Interactive graph cuts for optimal boundary & region segmentation of objects in nd images,” in Computer Vision, 2001. ICCV 2001. Proceedings. Eighth IEEE International Conference on, vol. 1. IEEE, 2001, pp. 105-112.
[5] C. Rother, V. Kolmogorov, and A. Blake, “Grabcut: Interactive foreground extraction using iterated graph cuts,” in ACM transactions on graphics (TOG), vol. 23, no. 3. ACM, 2004, pp. 309-314.
[6] V. Gulshan, C. Rother, A. Criminisi, A. Blake, and A. Zisserman,“Geodesic star convexity for interactive image segmentation,” in Computer Vision and Pattern Recognition (CVPR), 2010 IEEE Conference on. IEEE, 2010, pp. 3129-3136.
[7] B. L. Price, B. Morse, and S. Cohen, “Geodesic graph cut for interactive image segmentation,” in 2010 IEEE Computer Society Conference on Computer Vision and Pattern Recognition. IEEE, 2010, pp. 3161-3168.
[8] J. Ning, L. Zhang, D. Zhang, and C. Wu, “Interactive image segmentation by maximal similarity based region merging,” Pattern Recognition, vol. 43, no. 2, pp. 445-456, 2010.
[9] M. Jian and C. Jung, “Interactive image segmentation using adaptive constraint propagation,” IEEE transactions on image processing, vol. 25, no. 3, pp. 1301-1311, 2016.
[10] Y. Li, J. Sun, C.-K. Tang, and H.-Y. Shum, “Lazy snapping,” in ACM Transactions on Graphics (ToG), vol. 23, no. 3. ACM, 2004, pp. 303-308.
[11] G. Wang, W. Li, M. A. Zuluaga, R. Pratt, P. A. Patel, M. Aertsen, T. Doel, A. L. David, J. Deprest, S. Ourselin, et al., “Interactive medical image segmentation using deep learning with image-specific fine-tuning,” IEEE Transactions on Medical Imaging, 2018.
[12] D. Acuna, H. Ling, A. Kar, and S. Fidler, “Efficient interactive annotation of segmentation datasets with polygon-rnn++,” in Proceedings of the IEEE Conference on Computer Vision and Pattern Recognition, 2018, pp. 859-868.
[13] W. Li, Y. Shi, W. Yang, H. Wang, and Y. Gao, “Interactive image segmentation via cascaded metric learning,” in Image Processing (ICIP), 2015 IEEE International Conference on. IEEE, 2015, pp. 2900-2904.
[14] K. Rajendran, M. Walsh, N. De Ruiter, A. Chernoglazov, R. Panta, A. Butler, P. Butler, S. Bell, N. Anderson, T. Woodfield, et al., “Reducing beam hardening effects and metal artefacts in spectral ct using medipix3rx,” Journal of Instrumentation, vol. 9, no. 03, p. P03015, 2014.
[15] R. Achanta, A. Shaji, K. Smith, A. Lucchi, P. Fua, and S. S\ddot{ }usstrunk,“Slic superpixels compared to state-of-the-art superpixel methods,” IEEE transactions on pattern analysis and machine intelligence, vol. 34, no. 11, pp. 2274-2282, 2012.
[16] J. A. Hartigan and M. A. Wong, “Algorithm as 136: A k-means clustering algorithm,” Journal of the Royal Statistical Society. Series C (Applied Statistics), vol. 28, no. 1, pp. 100-108, 1979.
[17] M. Wang, X. Liu, Y. Gao, X. Ma, and N. Q. Soomro, “Superpixel segmentation: a benchmark,” Signal Processing: Image Communication, vol. 56, pp. 28-39, 2017.
[18] P. Neubert and P. Protzel, “Superpixel benchmark and comparison,” in Proc. Forum Bildverarbeitung, 2012, pp. 1-12.
[19] V. Gulshan, “From interactive to semantic image segmentation,” Ph.D. dissertation, University of Oxford, 2012.
[20] H. Zhou, J. Zheng, and L. Wei, “Texture aware image segmentation using graph cuts and active contours,” Pattern Recognition, vol. 46, no. 6, pp. 1719-1733, 2013.
[21] N. Houhou, J.-P. Thiran, and X. Bresson, “Fast texture segmentation based on semi-local region descriptor and active contour,” Numerical Mathematics: Theory, Methods and Applications., vol. 2, no. article, pp. 445-468, 2009.
[22] Z. Guo, L. Zhang, and D. Zhang, “A completed modeling of local binary pattern operator for texture classification,” IEEE transactions on image processing, vol. 19, no. 6, pp. 1657-1663, 2010.
[23] L. Liang, C. Liu, Y.-Q. Xu, B. Guo, and H.-Y. Shum, “Real-time texture synthesis by patch-based sampling,” ACM Transactions on Graphics (ToG), vol. 20, no. 3, pp. 127-150, 2001.
[24] N. Sochen, R. Kimmel, and R. Malladi, “A general framework for low level vision,” IEEE Transactions on Image Processing, vol. 7, no. 3, p. 310-318, 1998.
[25] H. Harzallah, F. Jurie, and C. Schmid, “Combining efficient object localization and image classification,” in Computer Vision, 2009 IEEE 12th International Conference on. IEEE, 2009, pp. 237-244.
[26] H. J\acute{ }egou, M. Douze, C. Schmid, and P. P\acute{ }erez, “Aggregating local descriptors into a compact image representation,” in Computer Vision and Pattern Recognition (CVPR), 2010 IEEE Conference on. IEEE, 2010, pp. 3304-3311.
[27] R. Arandjelovic and A. Zisserman, “All about vlad,” in Computer Vision and Pattern Recognition (CVPR), 2013 IEEE Conference on. IEEE, 2013, pp. 1578-1585.
[28] J. S\acute{ }anchez, F. Perronnin, T. Mensink, and J. Verbeek, “Image classification with the fisher vector: Theory and practice,” International journal of computer vision, vol. 105, no. 3, pp. 222-245, 2013.
[29] C. J. Burges, “A tutorial on support vector machines for pattern recognition,” Data Mining and Knowledge Discovery, vol. 2, no. 2, pp.121-167, June 1998.
[30] A. Krizhevsky, I. Sutskever, and G. E. Hinton, “Imagenet classification with deep convolutional neural networks,” in Advances in neural information processing systems, 2012, pp. 1097-1105.
[31] K Suzuki, I. Horiba, and N. Sugie, “Linear-time connected-component labeling based on sequential local operations,” Computer Vision and Image Understanding, vol. 89, no. 1, pp. 1-23, 2003.
[32] J. Kalpathy-Cramer and H. M\acute{ }uller, Systematic Evaluations and Ground Truth. Berlin, Heidelberg: Springer Berlin Heidelberg, 2011, pp. 497-520.

1. A computer-implemented method for segmentation of multi-energy CT data, the multi-energy CT data including data in three or more energy bands, the method including:
a) receiving in memory the multi-energy CT data;
b) displaying the multi-energy CT data to a user;
c) receiving user input of one or more region indicators for the displayed data;
d) dividing the data into superpixels;
e) labelling at least some of the superpixels based on the received region indicators;
f) constructing feature vectors for at least some of the superpixels;
g) based on the labelled superpixels and feature vectors, classifying the superpixels using a machine learning classifier; and
h) segmenting the data into two or more regions based on the classification of the superpixels.

2. The method of claim 1 wherein the machine learning classifier is a Support Vector Machine.

3. The method of claim 1, further including one or more of displaying the segmented data to a user and storing the segmented data in memory.

4. (canceled)

5. The method of claim 1 wherein dividing the data is achieved using Simple Linear Iterative Clustering.

6. The method of claim 1 wherein the superpixels are non-overlapping superpixels.

7. The method of claim 1 wherein classifying the superpixels includes classifying global superpixel level descriptors into different classes.

8. The method of claim 1 wherein constructing feature vectors for at least some of the superpixels is performed using a compact coding process.

9. The method of claim 1 wherein constructing feature vectors for at least some of the superpixels is performed using a Bag of Features, Bag of Words, Fisher Vectors or Vector of Locally Aggregated Descriptors (VLAD) process.

10. The method of claim 1 wherein constructing feature vectors for at least some of the superpixels includes encoding and pooling the feature vectors.

11. The method of claim 10 wherein the encoding includes clustering labelled superpixels to create a codebook of visual words.

12. The method of claim 10 wherein the encoding is performed using clustering techniques selected from k-means clustering, fuzzy clustering or gaussian mixture models.

13. The method of claim 11 wherein the pooling includes generating feature vectors using the codebook of visual words and augmented data from each superpixel.

14. (canceled)

15. The method of claim 1 wherein construction of feature vectors for a superpixel includes randomly sampling pixels from that superpixel.

16. (canceled)

17. The method of claim 1 including determining that the segmentation of the image is unacceptable, receiving user input of one or more further region indicators for the displayed data; labelling at least some of the superpixels based on the received further region indicators; based on the labelled superpixels and feature vectors, reclassifying the superpixels using the machine learning classifier; and resegmenting the image into two or more regions based on the reclassification of the superpixels.

18. The method of claim 1, including augmenting the image with one or more of: texture information, horizontal gradient information and vertical gradient information.

19. (canceled)

20. (canceled)

21. (canceled)

22. A computer-implemented method for segmentation of multi-energy CT data, the multi-energy CT data including a plurality of data elements in three or more energy bands, the method including:
a) receiving the multi-energy CT data in memory;
b) displaying the multi-energy CT data to a user;
c) dividing the multi-energy CT data into clusters of data elements;
d) receiving user input of one or more region indicators for the displayed data;
e) based on the received region indicators, labelling one or more of the clusters of data elements;
f) constructing feature vectors for at least some clusters of the data elements;
g) based on the feature vectors and labelled clusters of data elements, classifying the clusters of data elements; and
h) segmenting the image into two or more regions based on the classification of the clusters of data elements.

23. The method of claim 22 wherein the data elements are pixels or voxels.

24. The method of claim 22 wherein the clusters of data elements are superpixels or supervoxels.

25. A multi-energy CT method, including:
a. performing a CT scan using a multi-energy CT system using three or more energy bands, to produce multi-energy CT data; and
b. segmenting the multi-energy CT according to the method of claim 1.

26. (canceled)

27. (canceled)

28. A multi-energy CT system, including:
a multi-energy CT scanner configured to scan a subject to produce multi-energy CT data including data in three or more energy bands;
memory arranged to store the multi-energy CT data;
a display arranged to display the multi-energy CT data to a user;
a user input device arranged for user input of one or more region indicators for the displayed data; and
a processor arranged to:
a) divide at least some of the multi-energy CT data into superpixels;
b) label at least some of the superpixels based on the received region indicators;
c) construct feature vectors for at least some of the superpixels; and
d) based on the feature vectors and labelled superpixels, segment the image into two or more regions.
